(19) United States
(12) Patent Application Publication
(10) Pub. No.: US 2016/0346074 A1
(43) Pub. Date: Dec. 1, 2016

(54) Conical Vascular Filter Having a Web

(71) Applicant: THE REGENTS OF THE UNIVERSITY OF CALIFORNIA, Oakland, CA (US)

(72) Inventors: Bashir Tafti, Los Angeles, CA (US); Edward W. Lee, Los Angeles, CA (US); Stephen T. Kee, Santa Monica, CA (US)

(21) Appl. No.: 15/234,215

(22) Filed: Aug. 11, 2016

Related U.S. Application Data
(63) Continuation-in-part of application No. 15/074,766, filed on Mar. 18, 2016.
Application No. 15/074,766 is a continuation of application No. 14/585,795, filed on Dec. 30, 2014, now Pat. No. 9,289,280.
(60) Provisional application No. 62/014,334, filed on Jun. 19, 2014.

(51) Int. Cl. A61F 2/01

(52) U.S. Cl. CPC A61F 2/01; A61F 2002/011; A61F 2002/016; A61F 2002/018; A61F 2220/0016; A61F 2230/0076; A61F 2310/0097

(57) ABSTRACT

The present invention relates to a device for filtering obstructive material within the vasculature of a subject. The device includes a frame with multiple flexible legs connected to a hub, and a web that is positioned between the legs. The web is substantially perpendicular to the central axis. The legs are compressible about the central axis when the frame is in a compressed state, and the legs expand away from the central axis such that the web is held taut when the legs are in an expanded state. In certain embodiments, the web is a single piece of thread that passes through openings in the legs. In certain embodiments, a heat shrink material is used to secure a thread of the web to the frame.

### CROSS-REFERENCE TO RELATED APPLICATIONS

This application is a continuation-in-part of U.S. patent application Ser. No. 15/074,766, filed Mar. 18, 2016, which is a continuation of U.S. patent Ser. No. 14/585,795, filed Dec. 30, 2014 (now U.S. Pat. No. 9,289,280), and claims priority to U.S. Provisional Application No. 62/203,723, filed Aug. 11, 2015 and U.S. Provisional Application No. 62/014,334, filed on Jun. 19, 2014, all of which are incorporated herein by reference in their entireties.

### BACKGROUND OF THE INVENTION

It is estimated that each year, between 300,000 and 600,000 people in the United States are negatively affected by deep vein thrombosis (DVT) and pulmonary embolism (PE). Further, it is estimated that between 60,000 and 100,000 people in the United States die each year as a result of venous thromboembolism (VTE), a disease that includes both DVT and PE, and occurs when a blood clot breaks loose and travels in the blood towards the lungs. Patients who are at risk of developing DVT or PE but cannot undergo anticoagulation therapy due to bleeding complications or ineffectiveness may opt for a vascular filter implant as an alternative treatment. Patients undergoing surgery for blunt trauma, penetrating trauma, and falls also benefit from vascular filters. These filters, commonly called inferior vena cava (IVC) filters, capture dislodged blood clots from the inferior vena cava and iliac veins before they can reach the lungs and heart.

A typical IVC filter consists of several wire legs or struts arranged in a small conical shape. The filter is inserted into the IVC through either the jugular vein in the neck or the femoral vein in the groin, with the mouth of the cone facing towards the oncoming flow of blood. Barbs on the filter legs secure the filter to the internal walls of the vein, and the conical shape of the legs permits normal blood flow while capturing and holding loose blood clots and emboli.

After insertion, these filters may only be retrieved from one direction (the jugular or the femoral vein). Migration within the patient may cause the filter to tilt, positioning the retrieval hook in apposition to the blood vessel wall and out of reach of the filter retrieval device. The filter legs may also adhere to and even perforate the vessel wall, which may require an invasive surgical removal of the filter, increasing treatment costs and risk of complications to the patient. Further, a tilted filter changes the cross-sectional profile of the filter relative to the oncoming flow path of blood, which can lead to an inefficient and sub-optimal filter performance. Still further, some filters, such as the OPTEASE® IVC filter (Cordis Corp., Freemont, Calif., USA) have features at either pole that potentially push the incoming clot towards vessel walls and thereby increase the incidence of in-situ thrombus formation and filter occlusion. A recent attempt to create an improved retrievable IVC filter is the Crux® vena cava filter (Volcano Corp., San Diego, Calif., USA), which can be deployed and retrieved from either the jugular or femoral veins. However, the design of these types of filters leads to significant contact along the vessel wall and therefore does not minimize the problem of adhesions. Also, such elongated filters cannot be placed in patients with a short infrarenal IVC. Further, recent studies have also shown an increased incidence of DVT in patients with conventional filters, which may be linked to thrombotic occlusion of the filter leading to venous stasis upstream in the legs.

Further, numerous filter leg configurations are known in the art (see for example U.S. Pat. No. 8,628,556 to Tessmer, U.S. Pat. No. 8,361,103 to Weaver et al., U.S. Pat. No. 7,763,045 to Osborne, U.S. Pat. No. 6,436,120 to Meglin, U.S. Pat. No. 6391045 to Kim et al., U.S. Pat. No. 6,267,776 to O'Connell, U.S. Patent Publication No. 2014/0243878, U.S. Patent Publication No. 2014/0107694, U.S. Patent Publication No. 2010/0256669, and U.S. Patent Publication No. 2010/0152765). Generally, as mentioned above, it is desirable for filter legs to capture loose blood clots and emboli, while simultaneously minimizing flow impedance and blood flow turbulence. However, prior art filter designs that rely on filter legs for capturing loose blood clots and emboli have several disadvantages. First, increasing the number of leg and strut members can significantly decrease blood flow by increasing luminal impedance and adding to turbulent fluid dynamics within the vessel. In addition, conventional filter legs are typically made of a medical grade shape memory metal or alloy, such as Nitinol. Manipulating filter leg designs can significantly add to the weight and balance of the filter, compromising the ability for the filter to stay centered within the vessel. This can lead to a tilted or malpositioned filter, which will have suboptimal performance, is difficult to remove, and may otherwise cause physical harm to the patient while increasing treatment costs. Complex filter leg designs also have a larger collapsed profile, limiting advancement and retrieval of the filter to larger vessels.

Thus, there is need in the art for a removable IVC filter that is less likely to adhere to a vessel wall, can be bidirectionally deployed and retrieved, minimizes the occurrence of tilt after deployment, minimizes the risk of vessel perforation, may be adjustable during deployment, and minimizes the occurrence of thrombotic occlusion in the filter. Further, there is need in the art for a vascular filter that can reliably capture blood clot and emboli while facilitating laminar blood flow, without relying on a complex or cumbersome filter leg configuration. Embodiments of the present invention satisfies these needs.

### SUMMARY OF THE INVENTION

In one embodiment, a vascular filter device includes a frame having a proximal hub and at least three flexible legs connected to the proximal hub, where the proximal hub lies along a central axis of the frame; and a web that is positioned between the at least three legs and is substantially perpendicular to the central axis; where the legs are compressible about the central axis when the frame is in a compressed state, and where the legs expand away from the central axis such that the web is held taut when the legs are in an expanded state. In one embodiment, the web is substantially planar when the legs are in an expanded state. In one embodiment, the web includes a thread. In one embodiment, the web consists of a single thread. In one embodiment, the thread includes at least one of nylon, polyester, polyvinylidene fluoride and polypropylene. In one embodiment, the thread includes a biocompatible material that is flexible, elastic, or both. In one embodiment, the web includes multiple crossing segments. In one embodiment, the multiple crossing segments form multiple openings in the web. In one embodiment, each of the multiple openings are sized between 3×3 mm and 10×10 mm. In one embodiment, the multiple crossing segments are formed by a single thread. In one embodiment, multiple flexible legs each include first and second openings. In one embodiment, the first and second openings face the interior of the frame. In one embodiment, the web includes a thread that passes through each of the first and second openings in the multiple flexible legs. In one embodiment, one of the multiple flexible legs includes a heat shrink material. In one embodiment, the heat shrink material is configured to secure a thread of the web to the frame. In one embodiment, the frame is composed of a nonferromagnetic, flexible material. In one embodiment, the nonferromagnetic, flexible material is a shape-memory material. In one embodiment, the shape-memory material is Nitinol. In one embodiment, the frame fits within a catheter having a lumen of between about 3 F and 15 F when the frame is in a compressed state. In one embodiment, at least one of the flexible legs includes a barb. In one embodiment, the barb retracts into an opening of the flexible leg when the frame is in the compressed state. In one embodiment, the proximal hub includes a proximal hook. In one embodiment, the frame includes a biocompatible material on surfaces the frame. In one embodiment, the biocompatible material is drug-eluting. In one embodiment, the biocompatible material is a coating. In one embodiment, the biocompatible material is a heat-shrinking material.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a perspective view of a filter according to an exemplary embodiment.

FIG. 2 is a top view of the web element shown in FIG. 1.

FIG. 3 is a side view of the filter shown in FIG. 1 with the web element removed and the detail of the barbs shown.

FIGS. 4A-4C show various magnified perspective side views of barbs according to the embodiment of the filter shown in FIGS. 1-3.

FIG. 5 is a side view of a filter with an alternative web pattern according to an exemplary embodiment.

FIG. 6A is cross-sectional side view of a catheter delivery/retrieval system for filters according to various embodiments. FIG. 6B is a cross-sectional side view of a catheter delivery/retrieval system with a filter in a semi-compressed state.

FIG. 7 is a magnified view of a push rod engaging a retrieval hook for deployment of the filter.

FIG. 8A is a side view of a filter partially deployed in a body vessel according to an exemplary embodiment. FIG. 8B shows the filter fully deployed. FIG. 8C shows the filter snagged by a retrieval member.

FIG. 9 is a flow chart of a method of filter placement.

FIG. 10 is a flow chart of a method of filter retrieval. FIGS. 11A-11E show various views of a deployment system according to an alternate embodiment. FIG. 11A is a perspective view, FIG. 11B is a top view and FIG. 11C is a side view of the deployment system engaged with the filter. FIGS. 11D and 11E are cross-sectional diagrams showing the securement tab engaged and the securement tab disengaged respectively.

FIG. 12A is a side view of a filter according to an exemplary embodiment. FIG. 12B is a magnified view of openings on a leg of the filter shown in FIG. 12A, illustrating a thread pattern for the web according to an exemplary embodiment.

FIG. 13A is a magnified view of a starter filter leg on the filter shown in FIG. 12A, illustrating a thread pattern for the initial thread going in, the final thread coming out, and a heat shrinking material for securing the initial and final thread. FIG. 13B is a diagram of a thread weaving pattern for forming the web according to an exemplary embodiment.

FIGS. 14A-14C show various magnified perspective side views of barbs according to an embodiment of the filter.

### DETAILED DESCRIPTION

It is to be understood that the figures and descriptions of the present invention have been simplified to illustrate elements that are relevant for a clear understanding of the present invention, while eliminating, for the purpose of clarity, many other elements found in typical vascular filters. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range.

The present invention relates to a device for filtering obstructive material within the vasculature of a subject. As contemplated herein, the device may be used as an inferior vena cava (IVC) filter for the prevention of pulmonary embolism, or any other procedure requiring filtering of a vessel or vein.

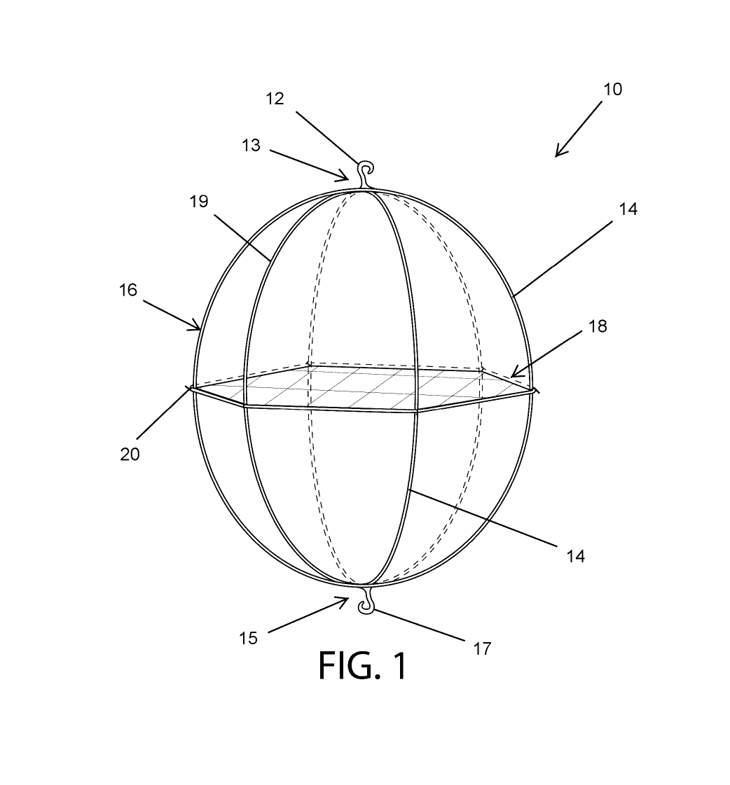

As shown in FIG. 1, the device 10 includes a frame 16 having a plurality of ellipses 14 connected at proximal base region 13 and distal base region 15. While there is no limitation to the number of ellipses 14, embodiments of the device 10 may include 2 ellipses, 3 ellipses, 4 ellipses, and even 5 or more ellipses. The ellipses 14 may be positioned equidistant from each other or they may be positioned variably such that the space between each is not uniform. Alternatively, the device 10 may be constructed as a frame 16 having a plurality of hemi-ellipses 19 connected at the proximal base region 13 and the distal base region 15. While there is no limitation to the number of hemi-ellipses 19 that could be used in the device 10, embodiments of device 10 may include 3 hemi-ellipses, 4 hemi-ellipses, 5 hemi-ellipses, 6 hemi-ellipses, and even 7 or more hemi-ellipses. Hemi-ellipses 19 may be positioned equidistant from each other or they may be positioned variably such that the space between each is not uniform. The filter 10 is bidirectional such that it is capable of equal filtering functionality of blood flow along the longitudinal axis in either the distal or proximal directions. Further, it is bidirectional in the sense that it can be advanced for delivery or retrieved for removal from either the proximal 13 or distal 15 sides of the device 10. The proximal 12 and distal 17 hooks are used to aid delivery and retrieval. They are preferably identical, however, it should be appreciated that there is no limitation to the size and/or shape of one or both of the hooks 12, 17.

In one embodiment, the frame 16 is composed of a nonferromagnetic, flexible, shape memory material, such as Nitinol. It should be appreciated that any rigid, yet flexible material may be used, such as a medical grade alloy or polymer, so that when the ellipses 14 or hemi-ellipses 19 are collapsed inwardly toward each other in a compressed state, an expanding bias is created, forcing ellipses 14 or hemi-ellipses 19 to return to their relaxed, expanded state. The medical grade materials described herein may also include an anti-thrombogenic coating or admixture to reduce the incidence of thrombus buildup, promoting hemocompatability and the maintenance of high blood flow rates through the filter.

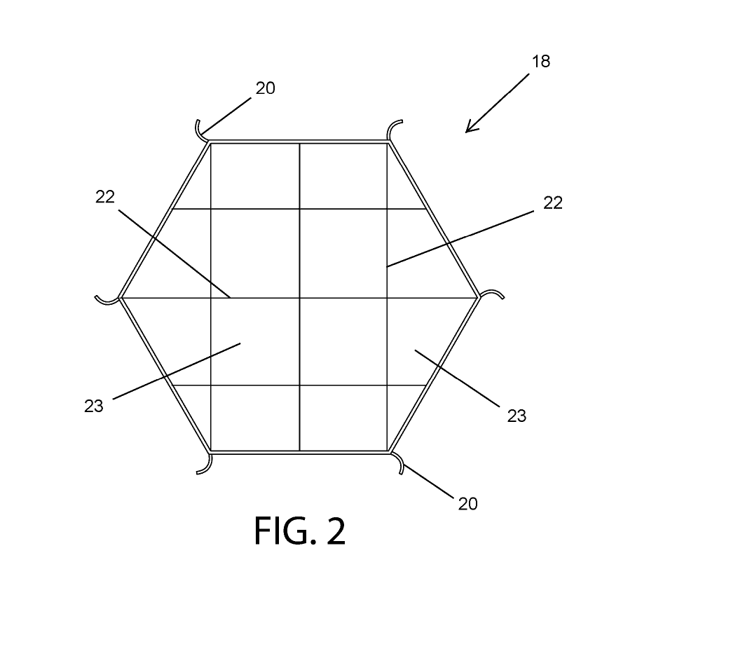

In a preferred embodiment, a web element 18 is positioned within the ellipses 14 of the frame 16. As shown in the exemplary embodiment of FIG. 2, the web element 18 forms a type of sieve, web or mesh-like feature for capturing blood clot or emboli traveling through the blood stream. The web element 18 may be positioned approximately half way between the proximal base region 13 and the distal base region 15. In a preferred embodiment, the web element 18 is positioned on a plane perpendicular to the longitudinal axis extending between the proximal end 13 and the distal end 15 of the filter 10. It should be appreciated that the web 18 may be positioned at any distance along the longitudinal axis between the proximal end 13 and the distal end 15 of the filter 10, and further, may be positioned at any angle within the arms of frame 16 suitable for capturing blood clot and emboli.

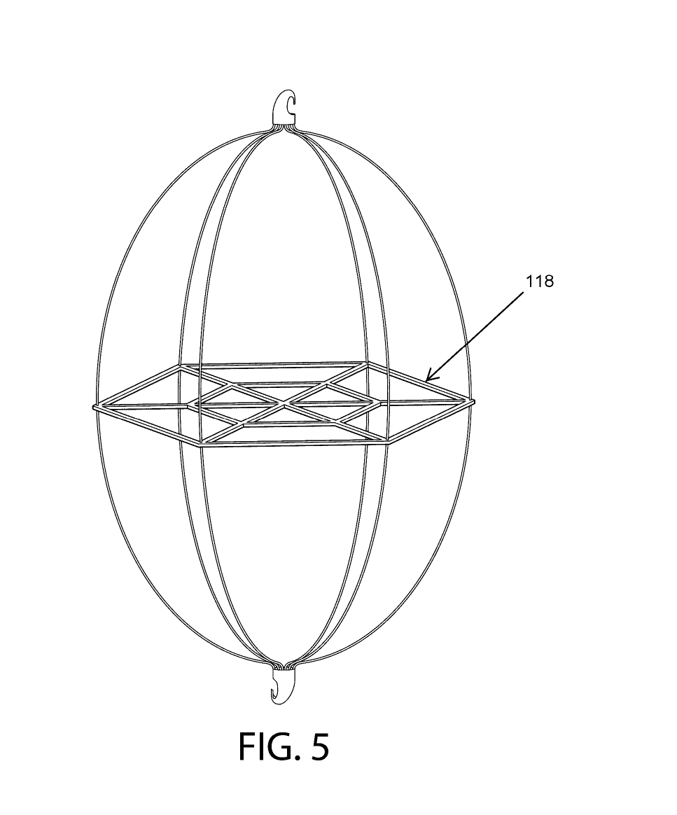

As shown in FIG. 2, the web element 18 includes a plurality of crossing fibers 22 that form a mesh-like structure of openings 23. As contemplated herein, the fibers 22 of the web element 18 may be separate from each other, partially connected or bonded to each other, or alternatively they may be molded as a single unit. The fibers 22 of the web element 18 may form a randomly patterned set of variable sized openings, or it may be geometrically patterned to form openings of a specific and uniform size in either a symmetrical or asymmetrical pattern. The web element pattern could be a grid-like pattern as shown in the web element 18 of FIG. 2, or it could be more of a concentric triangular and trapezoidal pattern as shown in the alternative web element embodiment 118 of FIG. 5. For example, in one embodiment, with reference back to FIG. 2, the openings 23 are quadrilaterals of about 6×6 mm in size. In preferred embodiments, the openings 23 are preferably any size between 3×3 mm and 10×10 mm, and further may approximate any shape fitting within those dimensions. The web element 18 may further be a single layer of material or it may be a multi-layered material, such that the desired filtering rate and blood flow rate though the vein is achieved. The web element 18 can also include an anti-thrombogenic property as described above. The fibers 22 of the web element 18 can be composed of an alloy, polymer, or any other biocompatible material that is rigid and flexible, and/or elastic. Exemplary and non-limiting materials for constructing the web include Nitinol, ePTFE, PTFE, and the like.

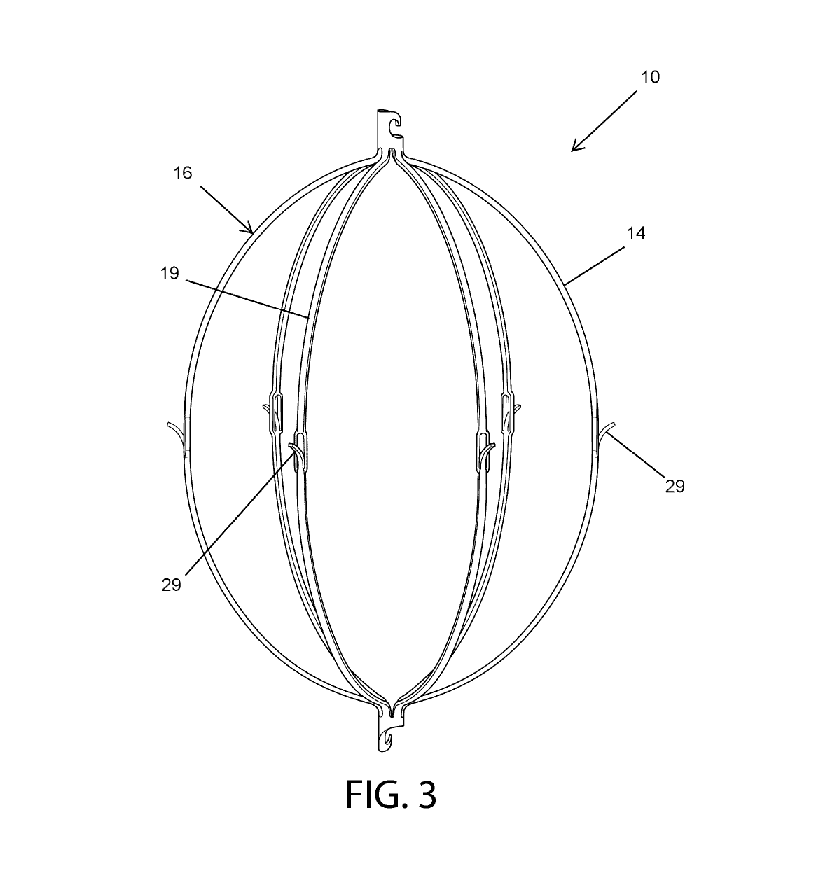

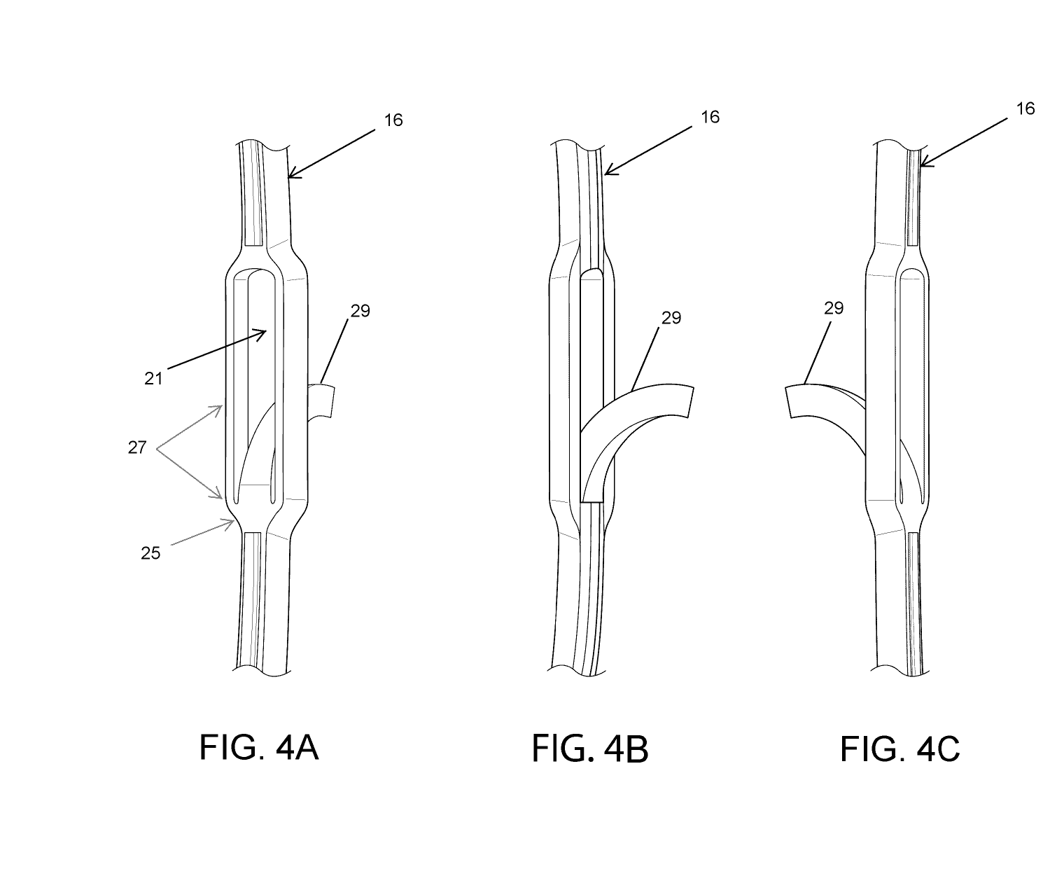

To prevent slippage of the filter 10 when positioned in a subject's vein and to anchor the device at a target treatment area, one or more barbs 29 may be positioned on a plane that bisects the filter's 10 longitudinal axis and runs along the minor axis of the ellipses 14 or the semi-minor axis of the hemi-ellipses 19 as shown in FIG. 3, or as shown with more detail in FIGS. 4A-4C. The barbs 29 secure and anchor the filter 10 against the patient's inner vessel wall as would be understood by those skilled in the art. Advantageously, since the barbs run along the path of the minor axis of the filter, a minimal footprint of contact with the inner vessel wall is achieved. The barbs can be on every ellipse along the minor axis as shown, or alternatively, can be present on alternate ellipses or any other combination of configurations. Barbs may project out from the filter perpendicularly, or otherwise be slanted in a proximal leaning or distal leaning direction. In alternate embodiments, barbs project out from the filter in a combination of perpendicular, proximally slanted, and distally slanted orientations. Barbs can also take a number of shapes, including curved, straight and variable thickness embodiments. With reference to the magnified views of FIGS. 4A-4C, the barbs are hinged at the bottom 25 of the openings 21, or at some portion 27 further up along the openings 21. The hinge acts as a strategic flex point so that while in a semi-collapsed or collapsed state, as the ellipses 14 collapse towards the center of the filter 10, the barbs 29 fold back about the flex point and into the openings 21, towards the middle of the device. In this state, the barbs remain tucked in below the outer surface of the filter 10. The hinge can be created structurally, for instance by the removal or reduction of framing 16 material (e.g. formation of the opening 21 itself), creating a weakened point of flexion along the frame 16 arm. Alternatively, the hinge can be created by a manufacturing step that incorporates a less rigid material at the desired flexion point, or by the introduction of additives that reduce material rigidity at the flexion point. Another method of forming the hinge includes a mechanical joint connecting two or more moving parts. Alternate embodiments do not have a hinge, and otherwise feature a contiguous member and composition of material along the length of the ellipse 14 and frame 16. Minimal exposure of the barbs above the surface of the filter 10 while in the semi-collapsed and collapsed states facilitates smooth advancement and retraction of the filter 10 during loading, placement and retrieval procedures. Further, the cross-sectional profile of the collapsed device is smaller and more spherical than conventional filters since the barbs tuck inward as opposed to being fixed and protruding out along outer surfaces of filter members. Advantageously, filters according to embodiments of the invention can fit into smaller delivery and retrieval catheters and devices, providing for minimized delivery and retrieval, and expanding treatment options for patients with a small or tortuous vein anatomy.

As shown in FIG. 1, the web element 18 can be coupled to the filter frame 16 at points along its circumference such that the web element lies along the minor axis of the filter 10. It should be appreciated that the web element 18 can be permanently secured to the frame 16, or it can be releasably secured to the frame 16 using methods known in the art or methods disclosed herein. For example, the web element 18 can include at least one hook or loop 20, as shown in FIG. 2, such that each hook or loop 20 is securely fastened to an arm of the frame 16. It should be appreciated that web element 18 can be coupled to only certain arms of the frame 16, and at any point along the length of the frame 16 arms, as desired. In another embodiment, the web element 18 is attached to the base of each barb 29 by fluorinated ethylene propylene (FEP) connections and secured by heat shrink processing.

In its relaxed state, the frame 16 expands the web element 18 so that each point of coupling between the web element 18 and the ellipses 14 or hemi-ellipses 19 holds the web element 18 substantially taut. When the frame 16 is compressed inwardly and towards the longitudinal axis of the filter 10, the web element 18 collapses within the frame 16 and assumes a much smaller profile, capable of sliding within the lumen of a delivery or retrieval device. The filter 10 can be sized so as to compress and collapse into a generally cylindrical conformation that fits within a standard catheter, sized to fit into a lumen of between about 3 F to 15 F. In one embodiment, the filter 10 is sized for use with a 6 F to 12 F catheter, and more preferably, a 6 F to 9 F catheter for delivery to or retrieval from the subject's vein.

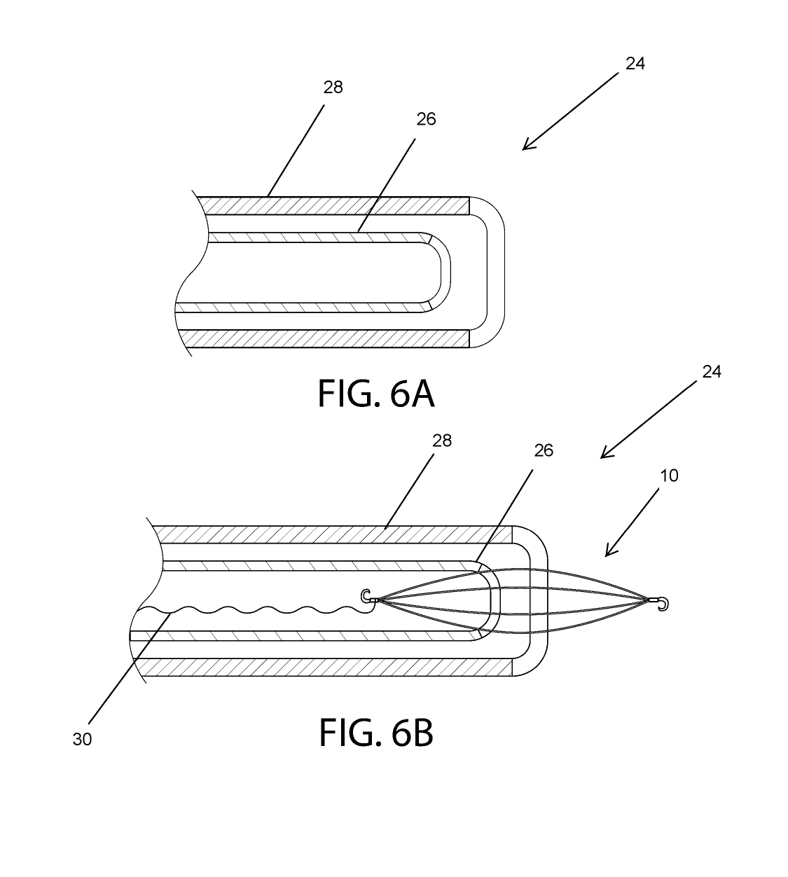

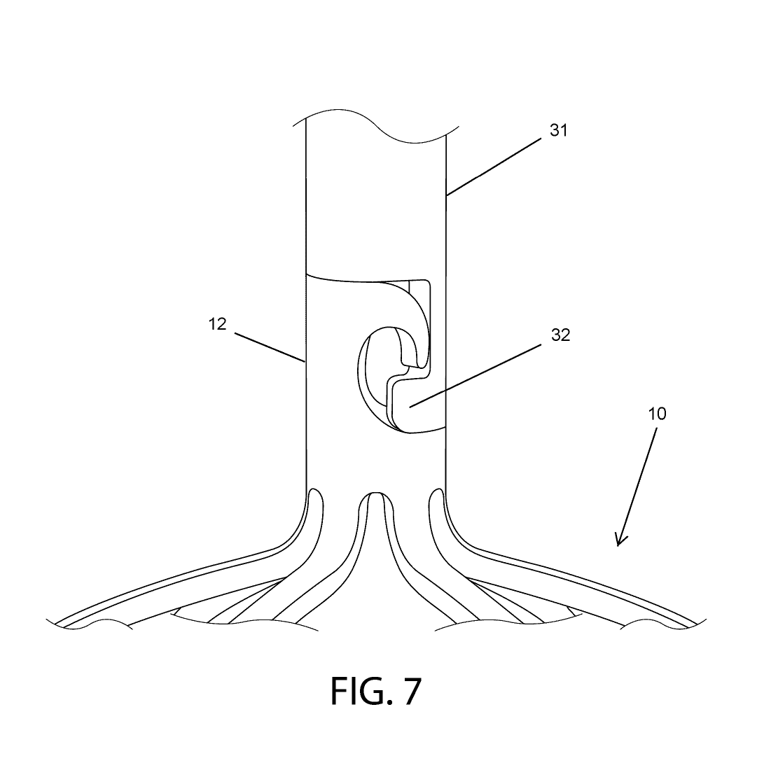

The filter 10 can be deployed into and retrieved from a subject's vein using a catheter-based system. As shown in the exemplary embodiment of FIG. 6A, the catheter system 24 includes an inner sheath 26 loaded within the lumen of an outer sheath 28. The filter 10 is compressed and collapsed into a thin cylindrical conformation such that it fits within the lumen of the inner sheath 26 as shown partially inserted in FIG. 6B. The filter 10 can be advanced forward by pushing the inner sheath 26 forward and pulled back by pulling on the snare 30. In a preferred embodiment and for ease of delivery, the filter 10 can be releasably coupled to a push rod 31 via the proximal hook 12 as shown in FIG. 7 (or alternatively the distal hook 17). In one embodiment, the push rod 31 may include a hook 32, loop, extension, or notch that engages with the proximal hook 12 as shown in FIG. 7. In this configuration, pushing on the push rod 31 may push the filter 10 out of the delivery sheath 26, expanding the filter 10 to its relaxed state and causing the perimeter of the filter 10 along the minor axis to engage the barbs 29 with the vessel wall, anchoring the filter at the point of treatment. Upon exiting the delivery sheath 26, the filter 10 can be decoupled from the push rod 31 by rotating push rod 31 along its longitudinal axis to disengage the notch 32 from the proximal hook 12. Alternatively, the push rod 31 need only contact an end of the filter 10, such that the filter 10 can be pushed out by the push rod 31 at the delivery site. In alternative embodiments, the filter 10 is deployable over a guidewire. Components at the proximal and distal ends of the filter 10, such as the proximal hook 12 and the distal hook 17, can include a guidewire lumen for loading the filter 10 over the guidewire. A retaining mechanism on the guidewire, such as a shaped section for forming an interference fit or other retaining mechanisms known in the art, can be used to secure the connection between the push rod 31 and the filter 10. During filter deployment within a vessel, once the filter 10 is advanced to a target position, the guidewire can be pulled back and retracted from its position within the filter 10, releasing the filter 10 from connection with the push rod 31, and allowing the push rod 31 to be withdrawn without dragging the filter from its target position. In certain embodiments, a vascular filter system includes a vascular filter device, having: a frame having multiple ellipses each having a major axis and a minor axis, with the major axes of each ellipse overlapping one another in a proximal and distal direction, a web positioned along its circumference to the minor axis of at least one ellipse, and a proximal hook coupled to the proximal end of the frame where the ellipses intersect at their proximal major axis vertices and a distal hook coupled to the distal end of the frame where the ellipses intersect at their distal major axis vertices; where the minor axes of the ellipses expand away from a central axis formed by their major axes, such that the web is held taut along its circumference when the ellipses are in an expanded state; and a guidewire; where the vascular filter device is configured to slidably load over the guidewire. The vascular filter device can have a guidewire lumen configured to coaxially load over the guidewire. The vascular filter system can also have an elongate deployment element and a retaining mechanism, wherein the retaining mechanism is configured to secure the elongate deployment element and the vascular filter device using the guidewire. The vascular filter system can also include an elongate deployment element and a retaining mechanism, where the retaining mechanism is configured to release the vascular filter device upon withdrawal of the guidewire from the vascular filter device.

When retrieving the filter 10, a conventional snare 30 can latch onto the proximal hook 12 or the distal hook 17 of the filter 10, as shown in FIG. 6B. In this configuration, maintaining tension on the snare 30 will hold the filter 10 stationary while the inner sheath 26 is slipped over the filter 10 to compress and release the filter 10 from the blood vessel wall. The filter 10 can then be completely retracted within the lumen of the inner sheath 26, and the inner sheath 26 can be retracted back within the outer sheath 28 for removal from the patient.

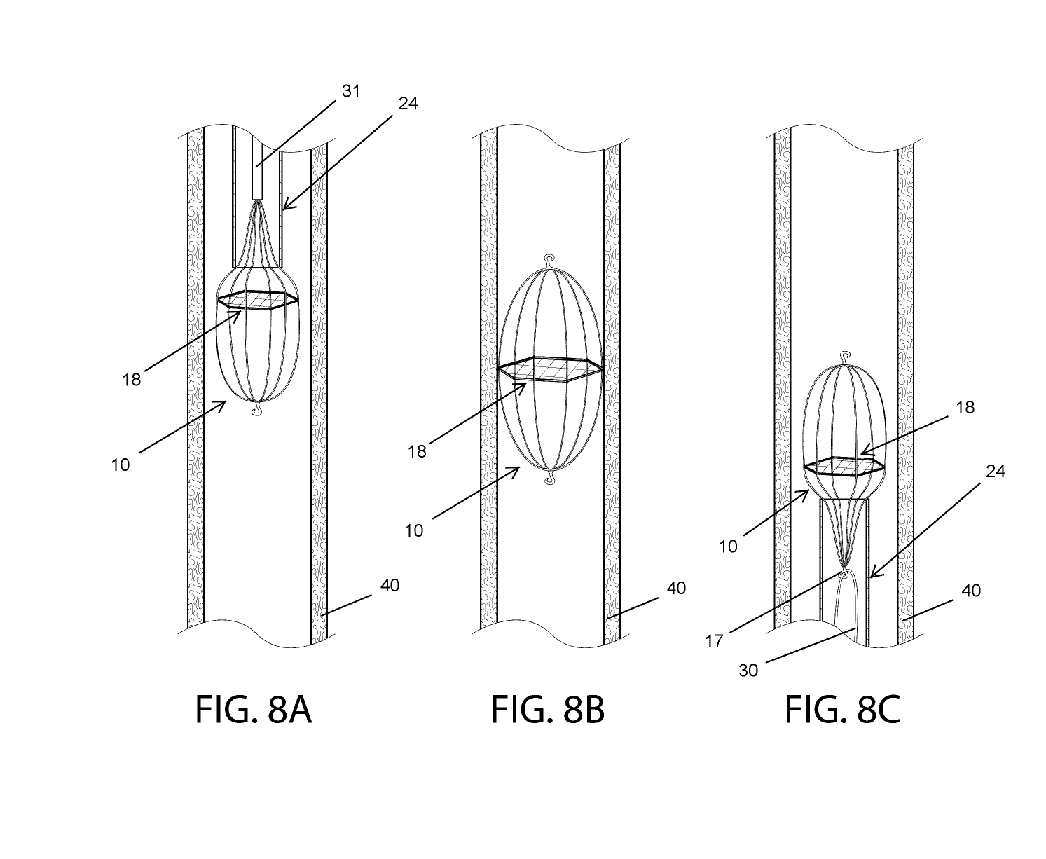

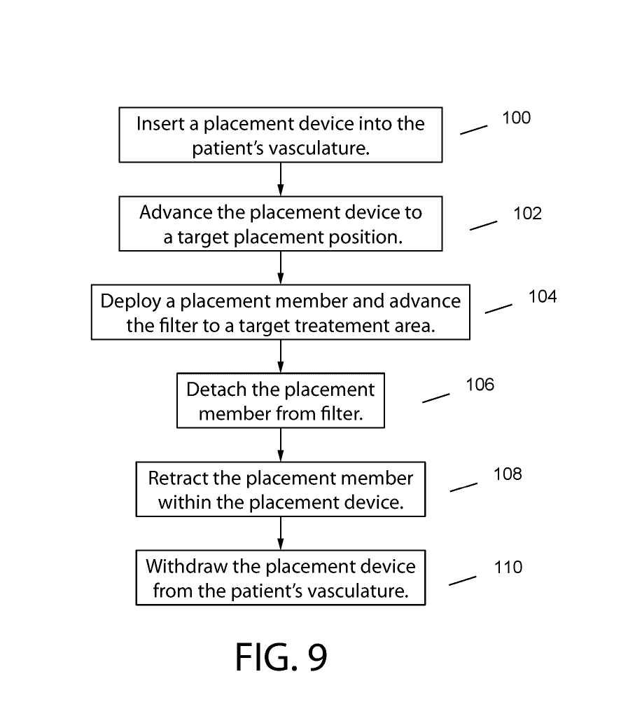

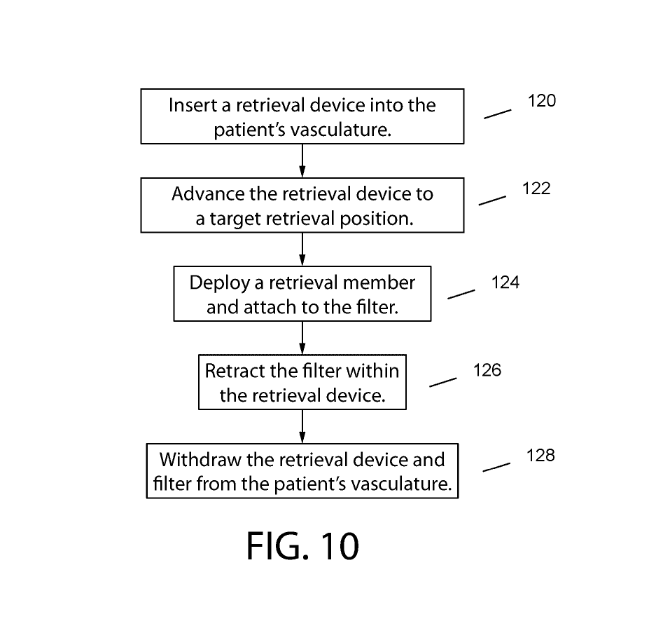

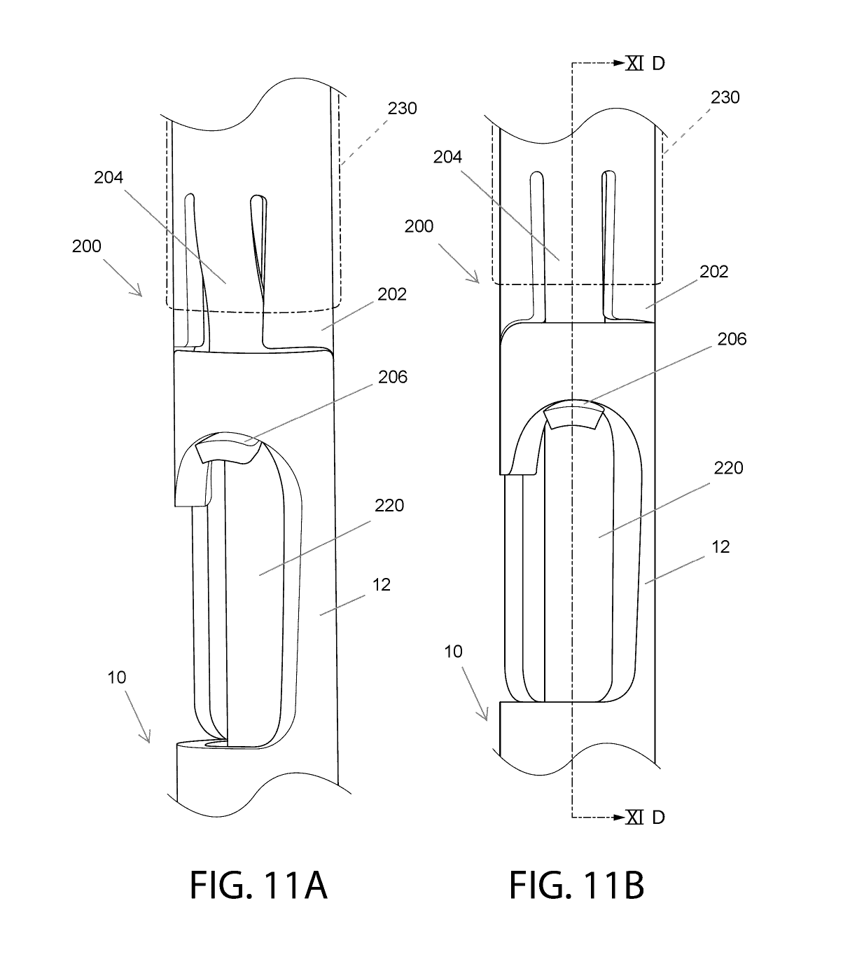

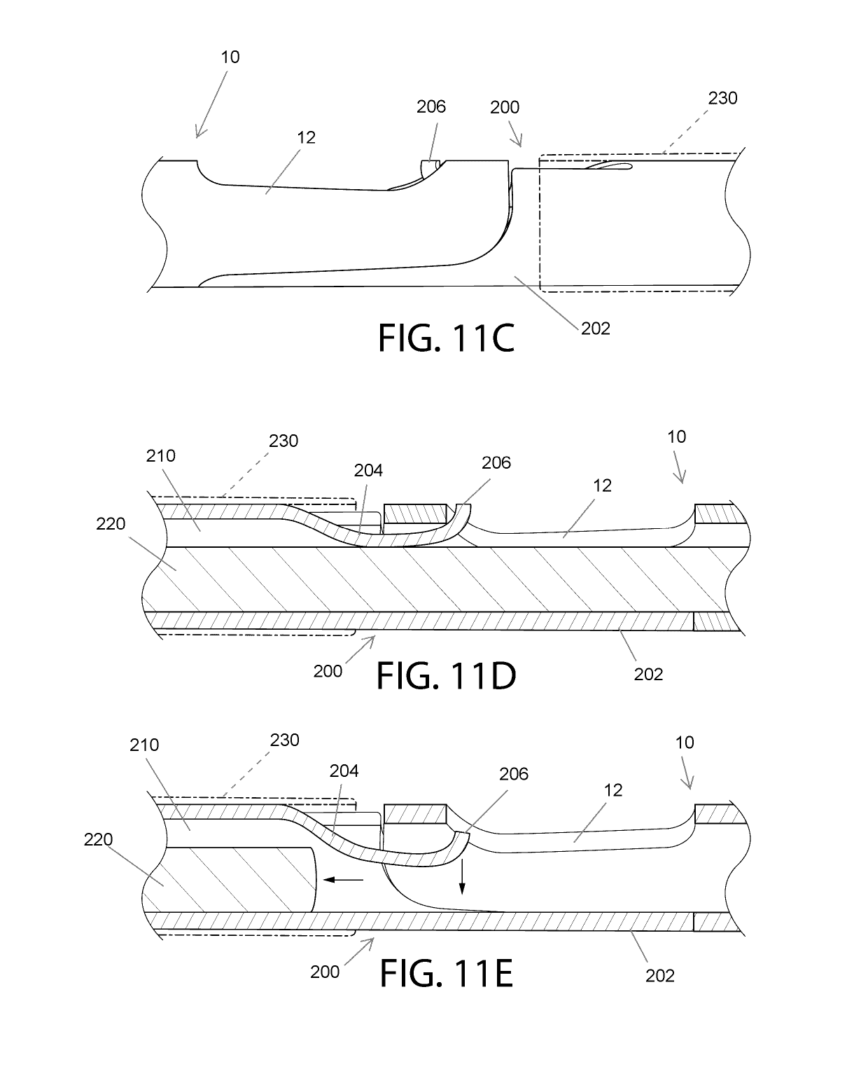

The advantages and improved performance of the filter 10 disclosed herein is further illustrated in FIGS. 8A-8C, with reference to the flow charts in FIGS. 9 and 10 outlining an exemplary method of treatment. A method of treatment starts with the insertion and placement of the filter 10 into the patient's vasculature 40 via the placement device 100. Placement devices described herein or known in the art can be used to place the filter 10. As shown in FIG. 8A, the placement device is advanced to a target placement position 102 within the patient's vessel 40, and the placement member (such as a push rod 31) can be deployed for advancing the filter to a target treatment area 104 as shown in FIG. 8B. Once the filter 10 is properly positioned, the placement member can be detached from the filter 106, retracted back into the placement device 108, and the placement device can be withdrawn from the patient's vasculature 110. The web element 18 is anchored perpendicular to the longitudinal axis of the vessel by barbs positioned at the minor axis of the ellipse as described above. This design is advantageous to maintaining a consistent perpendicular profile of the web element in relation to the oncoming flow of blood, providing a more predictable and reliable filtering mechanism that is not prone to tilt. The counterbalance of the filter 10 also helps to minimize any chance of the web element 18 tilting post insertion. To remove the device, the retrieval device is inserted back into the patient's vascular 120. Retrieval devices known in the art or as described herein can be utilized. Since the filter 10 is bidirectional, it can be snagged from either jugular or femoral veins. As illustrated in FIG. 8C, the retrieval device is advanced to the target retrieval position 122 from a femoral vein. A snare 30 is deployed that grabs on to the distal hook 17, 122. Once attached, the filter is retracted into the retrieval device 126 and the retrieval device is withdrawn from the patient's vasculature 128. When the filter is to be deployed via the femoral route, in a preferred embodiment, a special delivery sheath or catheter needs to be used. Such a catheter consists of a resistant but flexible inner lining (e.g. a Nitinol lining) that can withstand penetration by the filter while being flexible enough to navigate through the vessels.

An alternative embodiment of a deployment system 200 is shown in FIGS. 11A-11E. The deployment system 200 has a deployment element 202 designed to engage with a filter hook 12 (or 17) for securing the filter during advancement into a vessel. The deployment element 202 is geometrically opposed to the outer surfaces of the filter hook 12 such that the connection maintains a tight circular profile. This circular profile allows a procedural sheath 230 to slide over the connection. A tab 204 built into the deployment element 202 has a securement protrusion 206 that can mate to the back of the hook 12 (or 17). As shown in FIGS. 11A, 11B, 11D and 11E, a wire 220, such as a conventional guidewire or stylet, is present in the lumen 210 during deployment of the system. The tab 204 is naturally biased in a recessed position, towards the center of the deployment system lumen 210 as illustrated in FIG. 11E. When the wire 220 is introduced within the lumen 210 and the tab is advanced under the hook 12, the wire 220 will keep the tab 204 pushed up, securing the tab 204 and the deployment element 202 to the hook as shown in FIG. 11D. When the wire 220 is removed from the lumen 210, the tab 204 recesses back into its relaxed state within the lumen 210 as shown in FIG. 11E, disengaging from the hook 12. At this point, the deployment element 202 can retracted away from the hook 12 and removed from the vessel. The deployment element 202 can be made of materials including medical grade plastics known in the art, and manufactured using an injection molding process. In alternative embodiments, the tab actuates by a control that remains external during deployment, such as a tether or a powered control as known in the art.

In some embodiments, the filter 10 can be used in conjunction with one or more drug-eluting materials, such as the Translute™ drug carrying polymer (Boston Scientific Co., Natick, Mass., USA) or other commercially available drug-eluting materials as would be understood by those skilled in the art. For example, the frame of the device may be coated with a polymer carrying an anticoagulant, anti-fibrosis, or cytotoxin. In this embodiment, the device may release medication in a targeted fashion, thereby enhancing the ability of the device to prevent DVT and PE. In other embodiments, the device can be manufactured from or coated with polymer admixtures (e.g. fluoropolymers) that promote device hemocompatability.

The device of the above embodiments marks a significant improvement over current IVC filters. First, the bidirectional design of the filter reduces error of the filter being inserted in the wrong direction. Further, the symmetrical design and the presence of hooks at both proximal and distal ends allows for the deployment and retrieval of the device from either end. Further still, the inclusion of a web creates a single unit device to better capture smaller materials in the bloodstream without the use of secondary, loose components. In addition, the hooks or barbs along the circumference of the frame secures the device with fewer and less traumatic points of contact with a blood vessel wall to facilitate easier and less traumatic removal. Also, the filter is less prone to tilt, which increases the performance of the filter, increases the flow rate of blood through the filter, minimizing the chance that the patient will develop complications such as venous stasis downstream of the filter or thrombotic occlusion of the filter, further providing health professionals with a more accurate and predictable filtering rate.

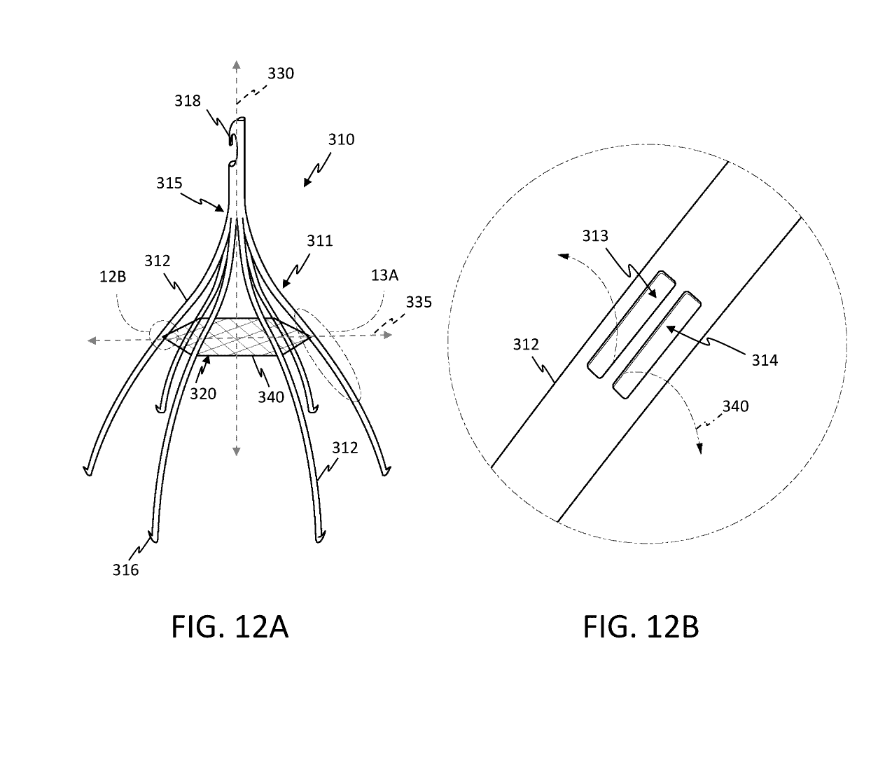

Now with reference to FIG. 12A, an embodiment of the filter 310 includes a frame 311 having a number of legs 312 that are connected to the proximal hub 315, extending distally away from the proximal hub 315. In certain embodiments, the proximal hub 315 lies along a central axis 330, which extends down through the center of the frame 311. The legs 312 generally have the shame shape and are spaced equidistant from each other, radially surrounding the central axis 330. While the legs 312 do not have to have the same geometry and spacing, certain embodiments use a common leg geometry and spacing to maintain a center of balance for the filter 310 along the central axis 330. While the embodiment of FIG. 12A shows 6 legs, it should be appreciated that the number of legs can include 3, 4, 5, 6, 7, 8, 9, 10 or more than 10 legs. The legs 312 converge onto the proximal hub 315, and can be formed using a number of methods known in the art. In certain embodiments, legs 312 attach to the hub 315 using adhesion or an electrical energy, or alternatively, the legs 312 are formed integral to the hub 315, through techniques including molding, laser fabrication, or formation from other known manufacturing techniques. In certain embodiments, the legs 312 generally curve away from the central axis 330 as they move distally away from the proximal hub 315. The proximal hub 315 can include a hook 318, a loop, or some other geometry that facilitates placement and/or retrieval of the filter. The geometry of the placement and/or retrieval element can also be configured to mate or interface with one or more types of placement or retrieval devices, such as forceps, a snare or a retrieval hook.

In certain embodiments, the frame 311 is composed of a nonferromagnetic, flexible material, or a medical grade shape memory material such as or Nitinol. The legs 312 are flexible, and can collapse in towards the central axis 330 when in a compressed state. In the relaxed state (see for example FIG. 12A), the legs 312 of the frame 311 expand away from the central axis 330. The filter 310 includes a web 320 that is positioned within the legs 312. As shown in the exemplary embodiment of FIG. 12A, the web element 320 forms a type of sieve, web or mesh-like feature for capturing blood clot or emboli traveling through the blood stream. In certain embodiments, the web 320 has a perimeter that has flat sides, or that are substantially circumferential, so that the web fits within the legs making up the frame 311. In certain embodiments, the web 320 is positioned approximately half way between the distal tips of the legs 312 and the proximal tip of the hub 315.

In certain embodiments, as shown in FIG. 12A, the web 320 is substantially planar when the legs 312 are in an expanded state. When the filter 310 is in the expanded state, the web 320 is held taught at or near its perimeter, which is connected to legs 312 of the filter 310. When the filter 310 is in the collapsed state, the web 320 will also collapse, since the web 320 is made of a biocompatible material that is flexible, elastic or both. In certain embodiments, the web 320 is made of thread 340. In certain embodiments, the web 320 is a single thread 340 that is weaved through openings 313, 314 in the legs 312 to form the web pattern. The thread 340 can be a number of materials known in the art, including nylon, polyester, polyvinylidene fluoride and polypropylene. Advantageously, since the filtering function is achieved primarily by the thread 340, and not the filter legs 312, filters according to the disclosed embodiments can collapse into a smaller profile, since the web 320 is flexible and easily collapsible, and since there is no need for a complex and cumbersome filter leg structure. The minimal emphasis on leg structure also allows the filter to be more flexible when collapsed during delivery, allowing the filter to be delivered and retrieved through smaller and more tortious vessel anatomies. In certain embodiments, the thread 340 is a blend of more than one material, such as a more durable material near the perimeter to prevent premature ware at connection points to the legs 312, and a more resilient material at portions of the web 320 within the web perimeter.

As shown with more detail in FIG. 12B, legs 312 of the frame 311 can include openings 313, 314 that in certain embodiments are first and second slots 313, 314, for passing or weaving a thread 340 through. In certain embodiments, at least one or both of the first and second openings 313, 314 in the legs 312 generally face towards the central axis 330 or the interior of the frame 311. In certain embodiments, the thread 340 is connected to the legs 312 by weaving the thread 340 into the first opening 313 and out of the second opening 340. In certain embodiments, the one or more openings in the leg are securement points for a terminal end of a particular segment of thread. In certain embodiments, the openings 313, 314 in the legs 312 lie along a common axis 335 that is substantially perpendicular to the central axis 330, so that when the frame 311 expands, the web 320 becomes substantially planar and perpendicular to the central axis 330. The web 320 includes a number of crossing segments that form a number of openings in the web 320. In certain embodiments, each of the plurality of openings are sized between 3×3 mm and 10×10 mm. The crossing segments can be formed by a single thread, or, for example, by 2, 3, 4, 5, or more than 5 threads that are secured to the legs 312. The thread 340 can form a randomly patterned set of variable sized openings, or it may be geometrically patterned to form openings of a specific and uniform size in either a symmetrical or asymmetrical pattern. The web 320 pattern could be a grid-like pattern, or it could be more of a concentric triangular and trapezoidal pattern. The web 320 may further be a single layer of material or it may be a multi-layered material, such that the desired filtering rate and blood flow rate though the vein is achieved. The web 320 can also include an anti-thrombogenic property as described herein. Although the embodiment shown in FIG. 12A shows the web 320 positioned perpendicular to the central axis, other embodiments may place openings 313, 314 of the legs 320 in such a position as to hold the planar web at an angle when the frame 311 is in an expanded state. Exemplary and non-limiting materials for constructing the web include Nitinol, ePTFE, PTFE (Polytetrafluoroethylene), and the like. In certain embodiments, the web is laser cut from a single monolithic piece, such as a single piece of flexible film or a single flexible sheet, and secured to the legs 320 using methods described herein, or other methods known in the art, such as the use of adhesives and electrical welding.

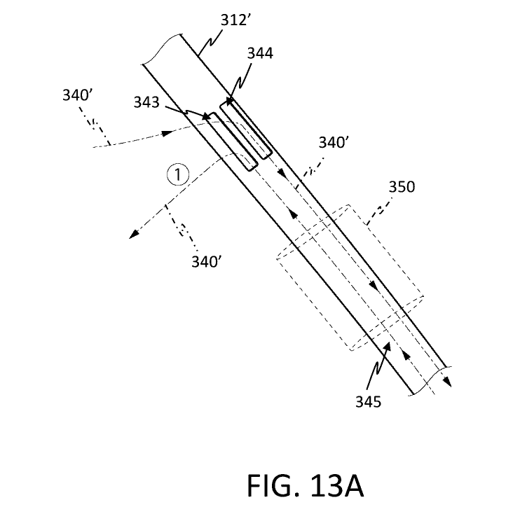

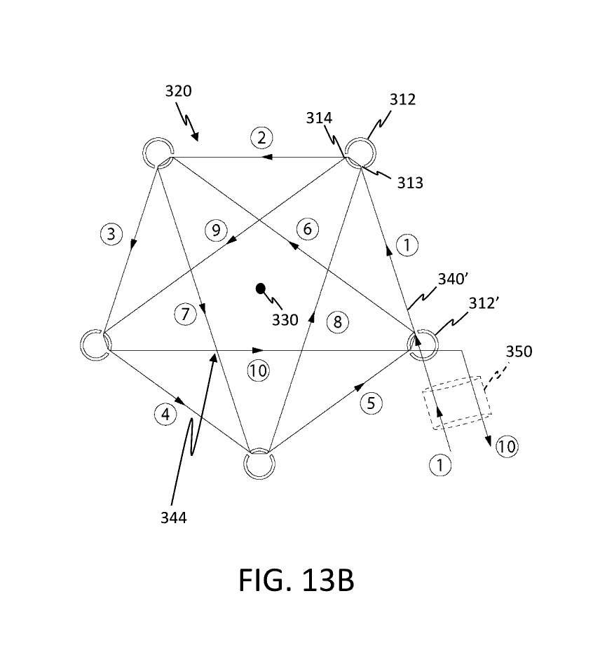

With reference now to FIGS. 13A and 13B, in one embodiment, a single thread 340′ is advanced through the lumen 345 of a starter leg 312′ which includes a heat shrink material 350. The starter leg 312′ is designed to capture and secure the beginning and ending of a single thread, to that the weaving pattern which forms the web can remain in place. The thread 340′ starts in a lumen 345 of the leg 312′, and surfaces out of the leg 312′ from a first opening 343. At that point, the surfaced thread 340′ is weaved through openings 313, 314 in other legs 312, similar to those openings described above for the various embodiments. The weaving pattern of the thread 340′ through the leg openings forms a web that in certain embodiments, matches the enumerated weaving order (steps 1-10) illustrated in FIG. 13B. In certain embodiments, the weave forms a radially symmetrical pattern around the central axis 330. In certain embodiments, the thread 340′ utilizes an over-under weaving pattern at crossover points 344 with other segments of the thread 340′. Crossover points 344 can also be bonded according to certain embodiments. When the weaving pattern is complete, the thread 340′ is reinserted into the second opening 344 of the starter leg 312′, and back down into the lumen 345 of the leg 312′. As mentioned above, a portion of the starter leg 312′ includes a heat shrink material 350. The heat shrink material 350 can shrink around the beginning and ending of the thread 340′, and under the application of heat, the heat shrink material 350 grabs onto the thread 340′ to secure the thread 340′ and the web 320 into place. It will be appreciated by those having ordinary skill in the art that this heat shrink material and leg opening feature is not limited to single thread web embodiments. The same starter leg configuration and heat shrink material configuration can be used to capture and secure the beginning and/or ending of any thread, including the ends of thread segments that makeup a multi-threaded web.

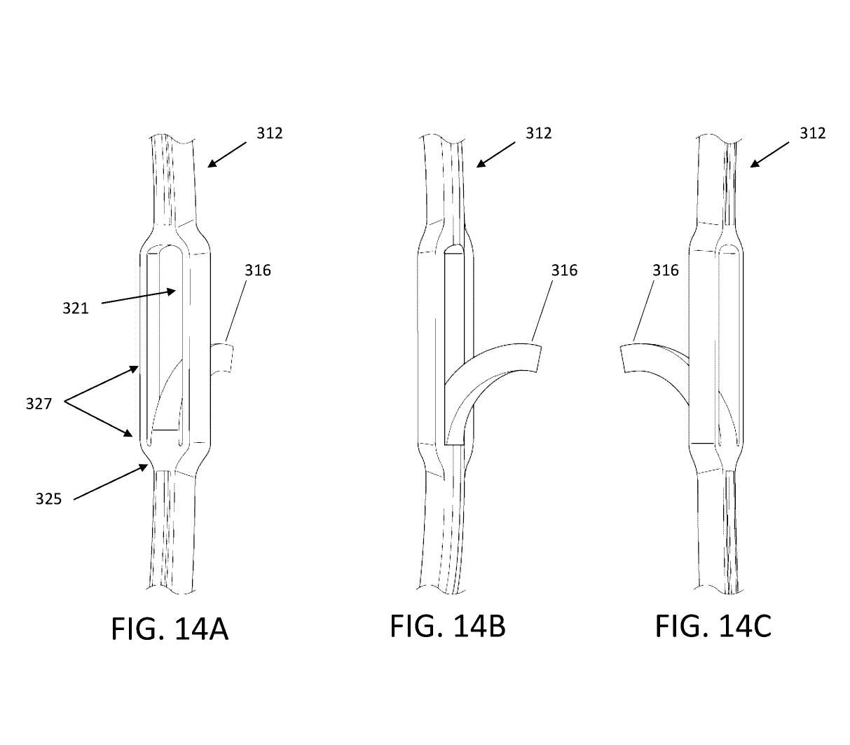

The frame 311 fits within a catheter lumen when the frame is in a compressed state. In certain embodiments, the catheter lumen is between about 3 F and 15 F, and is used for one or both of delivery and retrieval of the filter from a vessel in the patient, such as the inferior vena cava. In certain embodiments, one or more of the flexible legs 312 includes a barb 316 for securing the position of the filter 310 against a vessel wall. Barbs can take a number of shapes, including curved, straight and variable thickness embodiments. In one embodiment, one or more barbs 316 are positioned at the distal end of flexible leg 312, as shown in FIG. 12A. However, it should be appreciated that a barb 316 may be positioned at any point along the length of flexible leg 312. In another embodiment, a barb 316 may be retractable. For example, as shown in FIG. 14A-14C, the barbs 316 can be hinged at the bottom 325 of openings 321 or at some portion 327 further up along the openings 321. The hinge acts as a strategic flex point so that while in a semi-collapsed or collapsed state, as the flexible legs 312 collapse towards the center of the filter 310, the barbs 316 fold back about the flex point and into the openings 321, towards the middle or interior of the cone of the filter 310. In this state, the barbs 316 remain tucked in below the outer surface of the filter 310. The hinge can be created structurally, for instance by the removal or reduction of leg 312 material (e.g. formation of the opening 321 itself), creating a weakened point of flexion along the leg 312. Alternatively, the hinge can be created by a manufacturing step that incorporates a less rigid material at the desired flexion point, or by the introduction of additives that reduce material rigidity at the flexion point. Another method of forming the hinge includes a mechanical joint connecting two or more moving parts. Alternate embodiments do not have a hinge, and otherwise feature a contiguous member and composition of material along the length of the flexible leg 312. Minimal exposure of the barbs 316 above the surface of the filter 310 while in the semi-collapsed and collapsed states facilitates smooth advancement and retraction of the filter 310 during loading, placement and retrieval procedures. Further, the cross-sectional profile of the collapsed device is smaller than conventional conical filters since the barbs tuck inward as opposed to being fixed and protruding out along outer surfaces of filter legs. Advantageously, filters according to embodiments of the invention can fit into smaller delivery and retrieval catheters and devices, providing for minimized delivery and retrieval, and expanding treatment options for patients with a small or tortuous vein anatomy.

In the various embodiments described herein, including for example the embodiment shown in FIG. 12A, outer surfaces of the filter 310, such as the frame 311, may include a biocompatible material. The medical grade materials described herein may also include an anti-thrombogenic coating or admixture to reduce the incidence of thrombus buildup, promoting hemocompatability, patency and the maintenance of high blood flow rates through the filter. In certain embodiments, the biocompatible material is drug-eluting. In certain embodiments, the biocompatible material is a coating. In some embodiments, the filter 310 can be used in conjunction with one or more drug-eluting materials, such as the Translute™ drug carrying polymer (Boston Scientific Co., Natick, Mass., USA) or other commercially available drug-eluting materials as would be understood by those skilled in the art. For example, the frame of the device may be coated with a polymer carrying an anticoagulant, anti-fibrosis, or cytotoxin. In this embodiment, the device may release medication in a targeted fashion, thereby enhancing the ability of the device to prevent DVT and PE. In certain embodiments, the web is drug eluting, and can slowly release low doses of certain medications into the local environment. In certain embodiments, the drugs are anticoagulant medications (e.g. Plavix, Sanofi Corp., France), anti-proliferative medications (e.g. paclitaxel), or thrombolytic medications (e.g. tPA). In other embodiments, the device can be manufactured to include materials such as Nitinol or PTFE, coated with a biocompatible coating, or manufactured from a polymer admixture (e.g. fluoropolymers) that promotes device hemocompatability and reduce the risk of clot formation and fibrosis. In certain embodiments, such as when the filter is made of Nitinol, the filter's frame undergoes “Blue Oxide” surface finishing. In certain embodiments, the filter's frame is encased with heat-shrinking PFE tubing or a similar material, and is then heat treated. As a result the frame is tightly covered by PFE which in turn, reduces the risk of clot formation and fibrosis.

The device according to embodiments of the present invention marks a significant improvement over current filters. The inclusion of a web creates a single unit device to better capture smaller materials in the bloodstream without a heavy reliance on cumbersome filter leg structures, and without the use of secondary, loose components. Also, the filter is less prone to malposition and tilt. Overall, improvements of the filter according to the embodiments disclosed herein increase the performance of the filter, increase patency and the flow rate of blood through the filter, decrease the collapsed profile of the filter, increase the flexibility and maneuverability of the filter in the collapsed state, improve the laminar flow and fluid dynamics through the filter, minimize the chance that the patient will develop complications such as venous stasis downstream of the filter or thrombotic occlusion of the filter, and further provide health professionals with a more accurate and predictable filtering profile and filtering rate.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A vascular filter device comprising:
a frame having a proximal hub and at least three flexible legs connected to the proximal hub, wherein the proximal hub lies along a central axis of the frame; and
a web that is positioned between the at least three legs and is substantially perpendicular to the central axis;
wherein the legs are compressible about the central axis when the frame is in a compressed state, and wherein the legs expand away from the central axis such that the web is held taut when the legs are in an expanded state.

2. The device of claim 1, wherein the web is substantially planar when the legs are in an expanded state.

3. The device of claim 1, wherein the web comprises a thread.

4. The device of claim 3, wherein the web consists of a single thread.

5. The device of claim 3, wherein the thread comprises a biocompatible material that is flexible, elastic, or both.

6. The device of claim 1, wherein the web comprises a plurality of crossing segments.

7. The device of claim 6, wherein the plurality of crossing segments form a plurality of openings in the web.

8. The device of claim 7, wherein each of the plurality of openings are sized between 3×3 mm and 10×10 mm.

9. The device of claim 6, wherein the plurality of crossing segments are formed by a single thread.

10. The device of claim 1, wherein a plurality of the flexible legs each comprise first and second openings.

11. The device of claim 10, wherein the first and second openings face the interior of the frame.

12. The device of claim 10, wherein the web comprises a thread that passes through each of the first and second openings in the plurality of the flexible legs.

13. The device of claim 10, wherein one of the plurality of flexible legs comprises a heat shrink material.

14. The device of claim 13, wherein the heat shrink material is configured to secure a thread of the web to the frame.

15. The device of claim 1, wherein the frame is composed of a nonferromagnetic, flexible material.

16. The device of claim 1, wherein the frame fits within a catheter having a lumen of between about 3 F and 15 F when the frame is in a compressed state.

17. The device of claim 1, wherein at least one of the flexible legs comprises a barb that retracts into an opening of the flexible leg when the frame is in the compressed state.

18. The device of claim 1, wherein the frame comprises a biocompatible material on surfaces the frame.

19. The device of claim 18, wherein the biocompatible material is drug-eluting.

20. The device of claim 18, wherein the biocompatible material is a coating.
